(19) United States
(12) Patent Application Publication
(10) Pub. No.: US 2008/0181926 A1
(43) Pub. Date: Jul. 31, 2008

(54) Composites and methods of preparation and use thereof

(75) Inventors: Kalpana S. Katti, Fargo, ND (US); Devendra Verma, Fargo, ND (US); Dinesh R. Katti, Fargo, ND (US)

Correspondence Address: VIKSNINS HARRIS & PADYS PLLP, P.O. BOX 111098, ST. PAUL, MN, 55111-1098, US

(73) Assignee: NDSU Research Foundation, Fargo, ND (US)

(21) Appl. No.: 11/872,569

(22) Filed: Oct. 15, 2007

Related U.S. Application Data
(60) Provisional application No. 60/851,387, filed on Oct. 13, 2006.

(51) Int. Cl. A61L 27/40

(52) U.S. Cl. 424/423

(57) ABSTRACT

The invention provides composites, methods for their preparation, composites prepared according to the methods, and methods for using the composites.

### RELATED APPLICATION(S)

This patent document claims the benefit of priority of U.S. application Ser. No. 60/851,387, filed Oct. 13, 2006, which application is herein incorporated by reference.

### STATEMENT OF GOVERNMENT SUPPORT

This invention was made with government support under Grant #0132768 awarded by the National Science Foundation. The government has certain rights in the invention.

### BACKGROUND

Chitosan (poly-1,4-D-glucosamine) is a partially deacetylated derivative from chitin. Chitosan is a biodegradable, biocompatible, non-antigenic, and biofunctional polymer that is considered an excellent material for tissue regeneration. Its hydrophilic surface promotes cell adhesion, proliferation, and differentiation, and evokes minimal foreign body reaction on implantation. However, in spite of above mentioned favorable properties, low mechanical strength and loosening of structural integrity under wet conditions make chitosan unsuitable for bone tissue engineering. Thus, materials with improved properties are needed.

### SUMMARY OF CERTAIN EMBODIMENTS OF THE INVENTION

Accordingly, certain embodiments of the present invention provide composites that include chitosan, polygalacturonic acid, and hydroxyapatite.

Certain embodiments of the present invention provide methods for preparing a composite, including:

preparing a first mixture that includes chitosan and hydroxyapatite so as to form a composite that includes chitosan and hydroxyapatite;

preparing a second mixture that includes polygalacturonic acid and hydroxyapatite so as to form a composite that includes polygalacturonic acid and hydroxyapatite; and

combining the first and second mixtures to form a third mixture so as to form a composite that includes chitosan, polygalacturonic acid, and hydroxyapatite.

In certain embodiments of the invention, the hydroxyapatite in the first mixture is prepared by combining Na₂HPO₄ and CaCl₂ so as to form the hydroxyapatite.

In certain embodiments of the invention, the hydroxyapatite in the second mixture is prepared by combining Na₂HPO₄ and CaCl₂ so as to form the hydroxyapatite.

Certain embodiments of the present invention provide methods for preparing a composite, including preparing a mixture that includes chitosan, Na₂HPO₄ and CaCl₂ so as to form a composite that includes chitosan and hydroxyapatite.

Certain embodiments of the present invention provide methods for preparing a composite, including preparing a mixture that includes polygalacturonic acid, Na₂HPO₄ and CaCl₂ so as to form a composite that includes polygalacturonic acid and hydroxyapatite.

The methods of the invention may further include cross-linking the chitosan to the polygalacturonic acid.

The methods of the invention may further include separating the composite (e.g., a composite that includes chitosan, polygalacturonic acid, and hydroxyapatite, or a composite that includes chitosan and hydroxyapatite, or a composite that includes polygalacturonic acid and hydroxyapatite) from the mixture.

The methods of the invention may further include drying the composite.

Certain embodiments of the present invention provide composites prepared according to the methods of the invention.

Certain embodiments of the present invention provide composites that include chitosan, polygalacturonic acid, and hydroxyapatite.

Certain embodiments of the present invention provide pharmaceutical compositions that include a composite of the invention and a pharmaceutically acceptable carrier.

Certain embodiments of the present invention provide composites of the invention for use in medical treatment or diagnosis.

Certain embodiments of the present invention provide uses of a composite of the invention to prepare a medicament useful for treating a disease in an animal.

In certain embodiments of the invention, the animal is a mammal.

In certain embodiments of the invention, the mammal is a human.

### BRIEF DESCRIPTION OF THE FIGURES

FIG. 1: Monomer unit of (a) polygalacturonic acid and (b) chitosan.

FIG. 2: Schematic of synthesis method for ChiPgAHAP composites.

FIG. 3: XRD plots of (a) ChiPgAHAP (b) ChiHAP and (c) PgAHAP powder.

FIG. 4A: AFM-phase image of PgAHAP composite. FIG. 4B: AFM-phase image of ChiHAP composite. FIG. 4C AFM-phase image of ChiPgAHAP composite.

FIG. 5: Photoacoustic Fourier transform infrared spectra of (a) hydroxyapatite (b) PgA and (c) PgAHAP in the region of 4000-400 cm⁻¹ obtained at mirror velocity of 0.15 cm/s.

FIG. 6: Photoacoustic Fourier transform infrared spectra of (a) hydroxyapatite (b) Chitosan and (c) ChiHAP in the region of 4000-400 cm⁻¹ obtained at mirror velocity of 0.15 cm/s.

FIG. 7: Photoacoustic Fourier transform infrared spectra of (a) ChiHAP (b) chitosan (c) second derivative plot of ChiHAP spectrum and (d) second derivative plot of chitosan spectrum in the region of 1800-1200 cm⁻¹ obtained at mirror velocity of 0.15 cm/s.

FIG. 8: Photoacoustic Fourier transform infrared spectra of (a) PgAHAP (b) ChiHAP and (c) ChiPgAHAP in the region of 4000-400 cm⁻¹ obtained at mirror velocity of 0.15 cm/s.

FIG. 9: Photoacoustic Fourier transform infrared spectra (1800-700 cm⁻¹) of (a) ChiPgAHAP and (b) mathematically added (PgAHAP+ChiHAP) in the region of 1800-700 cm⁻¹ obtained at mirror velocity of 0.15 cm/s.

FIG. 10: Water absorbed by (a) ChiHAP and (b) ChiPgAHAP and (c) PgAHAP while soaked in SBF.

FIG. 11: Inverted light micrograph of mineral nodules on ChiPgAHAP composite films stained with alizarin red S. Positive red staining demonstrated the presence of calcium deposits, i.e., mineralization. Image was collected after 10 days from seeding cells.

FIG. 12: Inverted light micrograph of mineral nodules in ChiPgAHAP composite scaffold stained with a live/dead cell assay. Positive green staining showed the live cells. Image was collected after 21 days of seeding cells.

FIG. 13: SEM images of fibrous extracellular matrix synthesized by osteoblast cells on ChiPgAHAP composite films. Cells were fixed using glutaraldehyde after 18 days from seeding.

FIG. 14: SEM images of mineral nodules in ChiPgAHAP composite scaffold. Image shows proliferation of osteoblast cells and formation of matrix. Cells were fixed using glutaraldehyde after 21 days from seeding.

FIG. 15: The experimental X-ray diffraction plot (a-dots) of hydroxyapatite is superimposed over calculated plot (a-thick solid line). The difference plot (b) of hydroxyapatite is shown at the bottom.

FIG. 16: The experimental X-ray diffraction plot (a-dots) of PgAHAP is superimposed over calculated plot (a-Thick solid line). The difference plot (b) of PgAHAP is shown at the bottom.

FIG. 17: The experimental X-ray diffraction plot (a-dots) of ChiHAP is superimposed over calculated plot (a-Thick solid line). The difference plot (b) of ChiHAP is shown at the bottom.

FIG. 18: The experimental X-ray diffraction plot (a-dots) of ChiPgAHAP is superimposed over calculated plot (a-Thick solid line). The difference plot (b) of ChiPgAHAP is shown at the bottom.

FIG. 19: FIG. 19a depicts an AFM-phase image of PgAHAP50 composite. FIG. 19b depicts an AFM-phase image of ChiHAP50 composite. FIG. 19c depicts an AFM-phase image of ChiPgAHAP50 composite.

FIG. 20: Photoacoustic Fourier transform infrared spectra of (a) hydroxyapatite (b) PgA and (c) PgAHAP50 in the region of 4000-400 cm⁻¹ obtained at mirror velocity of 0.15 cm/s.

FIG. 21: Photoacoustic Fourier transform infrared spectra of (a) hydroxyapatite (b) Chitosan and (c) ChiHAP50 in the region of 4000-400 cm⁻¹ obtained at mirror velocity of 0.15 cm/s.

FIG. 22: Photoacoustic Fourier transform infrared spectra of (a) ChiHAP50 (b) chitosan (c) second derivative plot of ChiHAP50 spectrum and (d) second derivative plot of chitosan spectrum in the region of 1800-1200 cm⁻¹ obtained at mirror velocity of 0.15 cm/s.

FIG. 23: Photoacoustic Fourier transform infrared spectra of (a) PgAHAP50 (b) ChiGAP50 and (c) ChiPgAHAP50 in the region of 4000-400 cm⁻¹ obtained at mirror velocity of 0.15 cm/s.

FIG. 24: Photoacoustic Fourier transform infrared spectra (1800-700 cm⁻¹) of (a) ChiPgAHAP50 and (b) mathematically added (PgAHAP50+ChiHAP50) in the region of 1800-700 cm⁻¹ obtained at mirror velocity of 0.15 cm/s.

FIG. 25: Schematic showing amorphous phase in PgAHAP50, ChiHAP50 and ChiPgAHAP50 nanocomposites.

FIG. 26: SEM image of ChiPgA fibrous scaffold.

FIG. 27: SEM image of ChiPgAHAP composite fibrous scaffold.

FIG. 28: Osteoblast growth on ChiPgA and ChiPgAHAP composite scaffolds.

### DETAILED DESCRIPTION

Described herein are new composite materials. These composites, in certain embodiments, demonstrate very useful mechanical properties. These composites, in certain embodiments, may also demonstrate biocompatibility. One use for these composites is as a material for porous scaffolds, useful, e.g., for bone tissue engineering.

The composites can be biodegradable, biocompatible and nonantigenic because, e.g., of the biofunctional properties of chitosan. Additionally, these composites, in certain embodiments, improve chitosan's generally poor mechanical properties.

Accordingly, certain embodiments provide composites comprising chitosan, polygalacturonic acid, and hydroxyapatite. In certain embodiments, the composite is in the form of a fibrous scaffold. In certain embodiments, the fibers of the fibrous scaffold are about 1-2 μm in diameter. In certain embodiments, the composite is in the form of a film.

In certain embodiments, the composite further comprises osteoblast cells.

In certain embodiments, the composite further comprises calcium mineralization.

Certain embodiments provide methods for preparing a composite that comprises chitosan, polygalacturonic acid, and hydroxyapatite, comprising:

preparing a first mixture that comprises chitosan and hydroxyapatite so as to form a composite that comprises chitosan and hydroxyapatite;

preparing a second mixture that comprises polygalacturonic acid and hydroxyapatite so as to form a composite that comprises polygalacturonic acid and hydroxyapatite; and

combining the first and second mixtures to form a third mixture so as to form a composite that comprises chitosan, polygalacturonic acid, and hydroxyapatite.

In certain embodiments, the hydroxyapatite in the first mixture is prepared by combining Na₂HPO₄ and CaCl₂ so as to form the hydroxyapatite.

In certain embodiments, the hydroxyapatite in the second mixture is prepared by combining Na₂HPO₄ and CaCl₂ so as to form the hydroxyapatite.

In certain embodiments, the methods further comprise cross-linking the chitosan to the polygalacturonic acid.

In certain embodiments, the methods further comprise separating the composite that comprises chitosan, polygalacturonic acid, and hydroxyapatite, e.g., from the third mixture.

In certain embodiments, the methods further comprise drying the composite that comprises chitosan, polygalacturonic acid, and hydroxyapatite.

Certain embodiments provide methods for preparing a composite that comprises chitosan, polygalacturonic acid, and hydroxyapatite, comprising:

preparing a first mixture that comprises chitosan in deionized water;

preparing a second mixture that comprises polygalacturonic acid in deionized water;

combining the first and second mixtures to form a third mixture; and

combining a fourth mixture that comprises hydroxyapatite with the third mixture to form a fifth mixture so as to form a composite that comprises chitosan, polygalacturonic acid, and hydroxyapatite.

In certain embodiments, the methods further comprise sonicating the third mixture.

In certain embodiments, the methods further comprise freezing the fourth mixture.

In certain embodiments, the methods further comprise sonicating the fifth mixture.

Certain embodiments provide composites, e.g., that comprises chitosan, polygalacturonic acid, and hydroxyapatite, prepared according to a method described herein.

In certain embodiments, the composite is in the form of a fibrous scaffold. In certain embodiments, the fibers of the fibrous scaffold are about 1-2 μm in diameter. In certain embodiments, the composite is in the form of a film.

In certain embodiments, the composite further comprises osteoblast cells.

In certain embodiments, the composite further comprises calcium mineralization.

Certain embodiments provide methods for preparing a composite, comprising preparing a mixture that comprises chitosan, Na₂HPO₄ and CaCl₂ so as to form a composite that comprises chitosan and hydroxyapatite.

Certain embodiments provide methods for preparing a composite, comprising preparing a mixture that comprises polygalacturonic acid, Na₂HPO₄ and CaCl₂ so as to form a composite that comprises polygalacturonic acid and hydroxyapatite.

Certain embodiments provide methods for treating a patient having a damaged bone, comprising inserting into the patient a composite as described herein so as to treat the damaged bone.

In certain embodiments, the bone was damaged by an injury.

In certain embodiments, the bone was damaged by a disease.

In certain embodiments, the damaged bone is a portion of a joint.

Certain embodiments provide compositions comprising a composite as described herein and an acceptable carrier.

In certain embodiments, the carrier is a pharmaceutically acceptable carrier.

Certain embodiments provide composite as described herein use in medical treatment or diagnosis.

Certain embodiments provide the use of a composite as described herein to prepare a medicament useful for treating a disease or injury in an animal.

In certain embodiments, the disease or injury is a disease or injury of a bone.

In certain embodiments, the bone is a portion of a joint (e.g., the shoulder, hip or knee).

In certain embodiments, the animal is a mammal.

In certain embodiments, the mammal is a human (i.e., a male or a female).

In certain embodiments, these new materials may be formed into porous shapes using a variety of standard processing methods to make, e.g., scaffolds for replacement of bone in the case of injury or disease. Typically, in bone tissue engineering, such scaffolds are seeded with cells and inserted into the patient. Currently there is no such tissue engineered construct available for joint replacement. Joint replacement alternatives available today are implants, e.g., polymeric, ceramic and metallic. In certain embodiments of the invention, the composite is a powder.

Certain embodiments of the present invention describes the synthesis of new composite materials useful, e.g., for bone repair and replacement. Certain embodiments of the invention provide a synthesis method that is includes in situ precipitation of hydroxyapatite (HAP) with polygalacturonic acid (PgA) followed by composite processing with a chitosan (Chi) in situ mineralized hydroxyapatite. The composite preparation can be in solution. The prepared composite material can have high elastic modulus. The in situ preparation can provide an additional advantage for bioactivity as well as enhanced mechanical properties.

In situ mineralization methods for hydroxyapatite influences both mechanical property and bioactivity in hydroxyapatite composites that have applications as bone biomaterials. As described herein, in situ mineralization of hydroxyapatite in the presence of polyacrylic acid was performed. It is believed that a reason for the influence over the mechanical properties and bioactivity is from the influence over the polymer-mineral interfaces enabled by in situ fabrication.

As described herein, in situ mineralized hydroxyapatite was combined with polymers polygalacturonic acid and chitosan. It was hypothesized that the combination of calcium binding capabilities of PgA and mechanical properties of chitosan, as well as the advantage of good biocompatibity of both natural materials, would result in a superior composite material.

Thus, certain embodiments of the invention relate to compositions useful, e.g., in bone tissue engineering, e.g., as a scaffold, and as a bone paste additive. Certain embodiments of the invention also relate methods for producing the composites described herein.

The ratio(s) of the materials in the composite can be varied, e.g., from about 1:1 to about 2:1. In certain embodiments of the invention, the ratio(s) of a polymer(s) to mineral(s) is about 1:1. In certain embodiments of the invention, the ratio(s) of a polymer(s) to mineral(s) is about 1.5:1. In certain embodiments of the invention, the ratio(s) of a polymer(s) to mineral(s) is about 2:1. Altering the ratios can alter the elastic modulus and/or the hardness of the resulting composite. The ratios of each of the materials in a composite may be varied independently.

Certain embodiments of the present invention relate to chitosan-polygalacturonic acid-hydroxyapatite composites that are useful, e.g., for bone tissue engineering. Results related to the nano-mechanics, nano-structure and intermolecular interactions of the composites are presented herein. As described herein, the interfacial interactions between polygalacturonic acid, chitosan and hydroxyapatite have been studied by photoacoustic Fourier transform infrared (PA-FTIR) spectroscopy. The hydroxyapatite phase distribution and particle size have been investigated using AFM. The nano-mechanical response and swelling behavior have also been investigated.

Three composites; chitosan-hydroxyapatite (ChiHAP), polygalacturonic acid-hydroxyapatite (PgAHAP) and chitosan-polygalacturonic acid-hydroxyapatite (ChiPgAHAP) have been synthesized using an in situ mineralization process. In certain composites of the invention, the polymer to mineral ratio is maintained at about 1:1 and in ChiPgAHAP composites the chitosan to polygalacturonic acid (PgA) ratio is also about 1:1. Atomic force microscope phase imaging (AFM-PI) shows uniform distribution of hydroxyapatite nano-particles in polymer matrix. The average sizes of the particles in PgAHAP, ChiHAP and ChiPgAHAP were found to be about 25.2, 42.5 and 34.3 nm respectively. The intermolecular interactions between different components have been studied using Fourier transform infrared (FTIR) spectroscopy. While not a limitation of the present invention, the FTIR spectra indicate that in PgAHAP, polygalacturonic acid attaches to hydroxyapatite surface through dissociated carboxylate groups, whereas in ChiHAP, chitosan interacts with hydroxyapatite through amino groups. While not a limitation of the present invention, the FTIR results also indicate that in ChiPgAHAP composites, chitosan and PgA form complex bonds at interface. The nano-mechanical properties were determined using nanoindentation and elastic moduli of PgAHAP, ChiHAP and ChiPgAHAP composites were found to be 29.81, 17.56 and 23.62 GPa respectively and hardness values of 1.56, 0.65 and 1.14 GPa were obtained for three composites respectively.

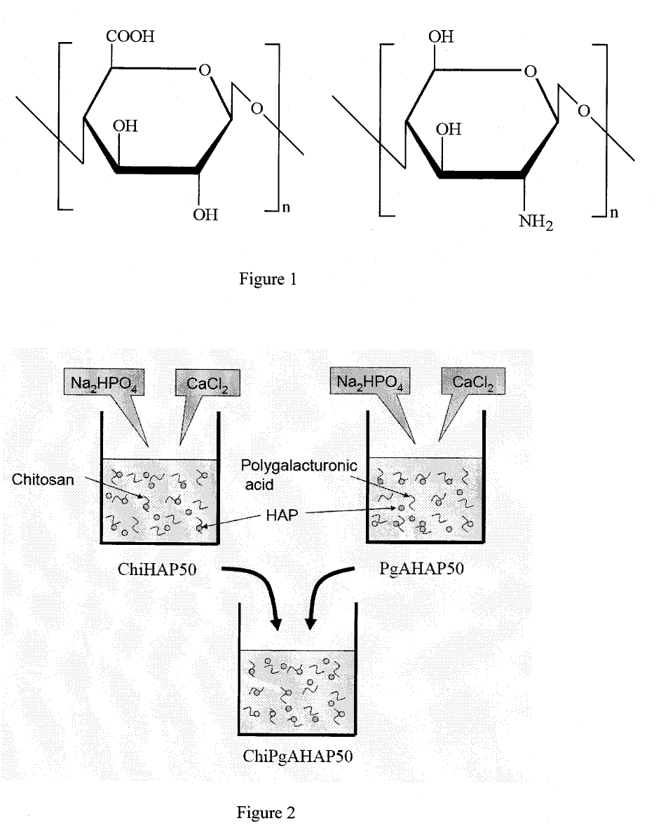

Pectin is a plant polysaccharide primarily obtained from edible plants. Pectin contains poly(d-galacturonic acid) bonded via glycosidic linkage. Pectin also contains neutral sugars, which are either inserted in or attached to the main chains. In pectin, the polygalacturonic acid is partly esterified with methyl groups. Pectin has gained increasing research interest as a drug carrier for oral drug delivery. Pectin has also been investigated for bone biomedical application and shown to improve cell adhesion and proliferation. Since pectin and chitosan are electrostatically complementary, they combine together in solution to form intermolecular complex. This complex has lower water solubility and improved mechanical response. Different interactions such as Van der Waals, electrostatic, hydrogen coordination bonding can occur between chitosan and pectin. The major interaction between chitosan and pectin occur is electrostatic interaction through amino and carboxylate groups. Polygalacturonic acid (PgA), de-esterified pectin is expected to have enhanced intermolecular interaction with chitosan because of higher content of free carboxylate groups. The monomer unit of PgA is shown in FIG. 1. In this study, chitosan-PgA-HAP composites have been synthesized by in situ mineralization method.

In situ mineralization is a biomimetic process in which mineralization occurs in close association with the polymer. Recently, this process has attracted much attention for composites design primarily for two reasons: first, to understand the fundamental knowledge behind biomineralization and second, for development of new materials with tailored structure and properties (see, e.g., Katti et al., Proc. 15th ASCE Engineering Mechanics Conf. New York, N.Y., 2002; Katti et al., Materials Research Society Proceeding, Boston, Mass., 711: GG4.3.1.-GG4.3.6, 2002; Verma et al., J Biomed Mater Res. 2006; 77A:59-66; Verma et al., J Biomed Mater Res, 78A, 772-780, 2006; Katti et al., American Journal of Biochemistry and Biotechnology, 2(2), 73-79, 2006; Mann et al., Science 1993; 261:1286-1292; Kato et al., Journal of Materials Science 1997; 32:5533-5543; Dalas et al., Langmuir 1991; 7:1822-1826; Katti, Colloids and Interfaces B 2004; 139:133-142; Verma et al., Materials Science and Engineering C, 2007, doi:10.1016/j.msec.2007.04.026; and Bhowmik et al., Materials Science and Engineering C, 27(3), 352-371, 2007).

Certain embodiments of the present invention will now be illustrated by the following non-limiting Examples.

### EXAMPLE 1

As described herein, nano-mechanical and nano-structural analysis of biomimetically synthesized chitosan-PgA-hydroxyapatite composites have been performed. These experiments are described in Verma et al., Materials Science and Engineering C, 2007, doi:10.1016/j.msec.2007.04.026, which publication is incorporated by reference herein as containing exemplary embodiments of certain aspects of the invention.

Interfacial interaction study has also been conducted. AFM-phase images show formation of nano-sized hydroxyapatite particles. However, Scherrer's analysis of XRD data indicated significantly lower crystallite sizes especially, in ChiHAP and ChiPgAHAP composites. From these observations it is believed that HAP particles are either made of multiple grains or contain significant amount of amorphous phase. The FTIR results indicate unidentate chelation of carboxylate groups of PgA, whereas, chitosan interacts with hydroxyapatite through amino groups. FTIR results also indicate interaction between carboxylate groups and amino groups in ChiPgAHAP composites. The ChiPgAHAP composites have shown improved mechanical response and maintained their structural integrity under SBF conditions.

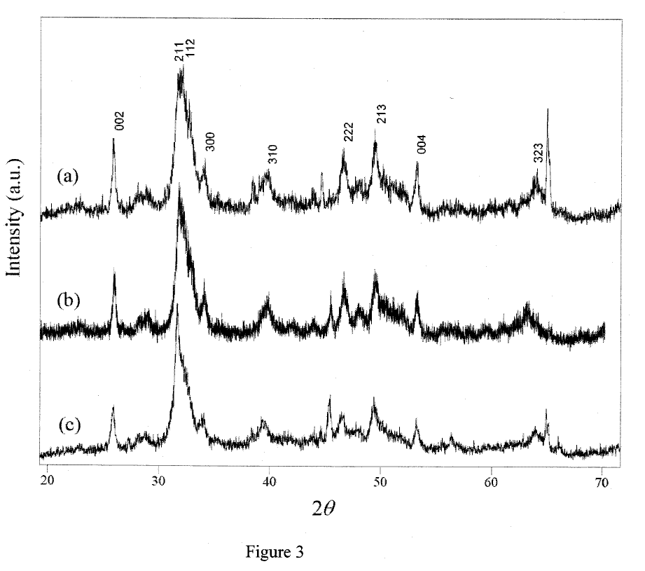

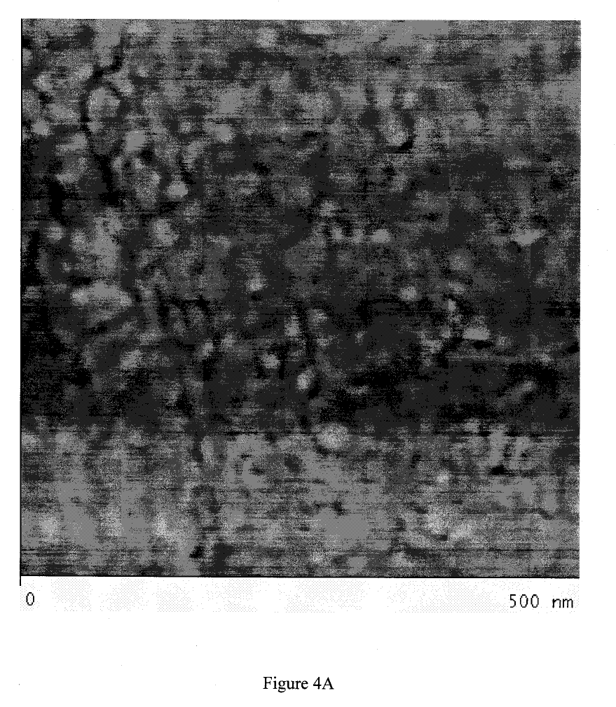

FIG. 3 shows x-ray diffraction (XRD) plots of PgAHAP, ChiHAP and ChiPgAHAP powder samples. XRD plots were compared with the Joint Committee for Powder Diffraction Studies (JCPDS) standard (09-0432). All samples show characteristic peaks of HAP. The crystallite sizes were determined using Scherrer's equation. For this purpose, the (0 0 2) peak was used. The crystallite sizes for PgAHAP, ChiHAP and ChiPgAHAP was found to be about 23 nm, 29 nm and 25 nm respectively. For these calculations peak broadening due to instrument and the lattice strain have not been taken into account.

### Nanostructure Analysis Using Atomic Force Microscopy (AFM)

In tapping mode AFM, the tip is oscillated at a frequency near its resonance and tip is allowed to make contact with the sample only for a short duration in each oscillation cycle. During oscillation of tip over sample surface, the tip-sample interaction may alter the amplitude, resonance frequency, and phase angle of the oscillating cantilever. Detection of phase angle changes of the cantilever probe during scanning provides an image, called as phase image. The phase angle change is associated with energy dissipation during sample-tip interaction. There are number of parameters that can cause energy dissipation, e.g., topography of the sample, sample-tip interactions, deformation of sample-tip contact area, and experimental conditions. The phase image is very useful for compositional mapping of surfaces and interfaces of polymeric materials and generally provides better contrast than the topographic images.

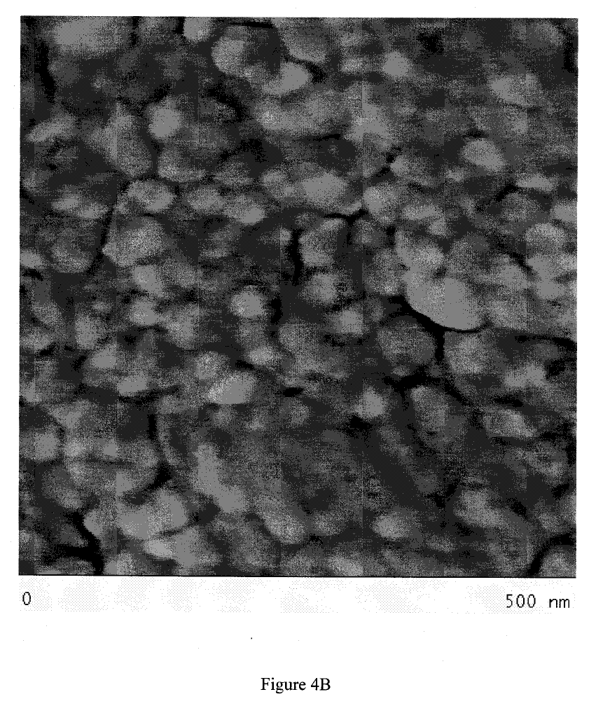

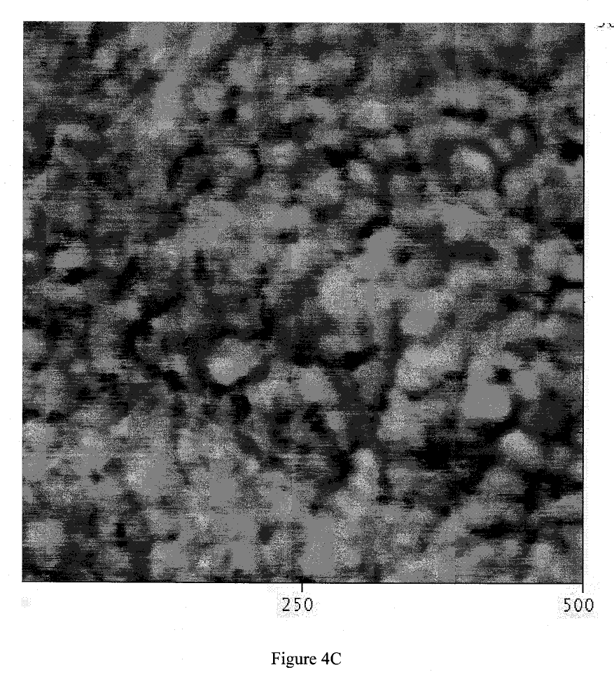

FIG. 4 (A, B, C) shows AFM-phase images of PgAHAP, ChiHAP, and ChiPgAHAP composites. The HAP and polymer phases are clearly distinguishable from AFM-phase images. In PgAHAP composite, the average HAP particle sizes was found to be about 25 nm, whereas in ChiHAP, and ChiPgAHAP composites particle sizes of 43 and 34 nm were obtained respectively. These differences in particle sizes in the composites can be attributed to effect of polymers on mineralization of hydroxyapatite. The role of polymers on mineralization has been focus of many studies. Various polymers, both of biological and synthetic origin, with different functionalities have been used to understand fundamental knowledge governing biomineralization. A polymer may cause acceleration or inhibition of crystal growth depending on its functionality, molecular weight, concentration, density of functional groups on the backbone chain or side chain, and whether polymer is adsorbed on surface or present in solution (Tsortos et al., Journal of Colloid and Interface Science 2002; 250:159-167). These functional groups have high affinity to bind to calcium ions on growth surface or in solution. While not a limitation of the present invention, it is believed that the smaller crystal sizes of hydroxyapatite in PgAHAP could be attributed to inhibitory effect caused by carboxylate groups of polygalacturonic acid. The crystallite sizes calculated from XRD plots have lower values compared to particle sized determined from AFM-phase images. While not a limitation, this suggests that HAP particles observed from AFM-phase images are composed of multiple grains or may contain significant amount of amorphous phase, especially in case of chiHAP and ChiPgAHAP. The growth of hydroxyapatite particles in presence of chitosan or PgA is similar to biomineralization process, where proteins and other polymers control the growth of the mineral.

### Nano Mechanical Properties

Nanoindentation tests were conducted using a diamond berkovich tip. Load controlled indentation tests were performed at a 5-second loading followed by holding for 5 seconds and then unloading for 5 seconds. All indentations were performed at load of 1000 μN. The average value of elastic moduli and hardness are given in Table 1. The average value of elastic moduli of PgAHAP, ChiHAP and ChiPgAHAP composites were found to be about 29.81, 17.56 and 23.62 GPa respectively. The average value of hardness of PgAHAP, ChiHAP and ChiPgAHAP were found to be about 1.56, 0.65, and 1.14 GPa respectively.

TABLE 1
Elastic modulus and hardness of PgAHAP, ChiHAP and ChiPgAHAP
composites as determined from nanoindentation experiments.

| | Elastic | Standard | | Standard |
|---|---|---|---|---|
| | Modulus | Deviation | Hardness | Deviation |
| | (GPa) | (GPa) | (GPa) | (GPa) |
| PgAHAP | 29.81 | 4.76 | 1.56 | 0.36 |
| ChiHAP | 17.56 | 0.93 | 0.65 | 0.21 |
| ChiPgAHAP | 23.62 | 4.03 | 1.14 | 0.34 |

### PA-FTIR

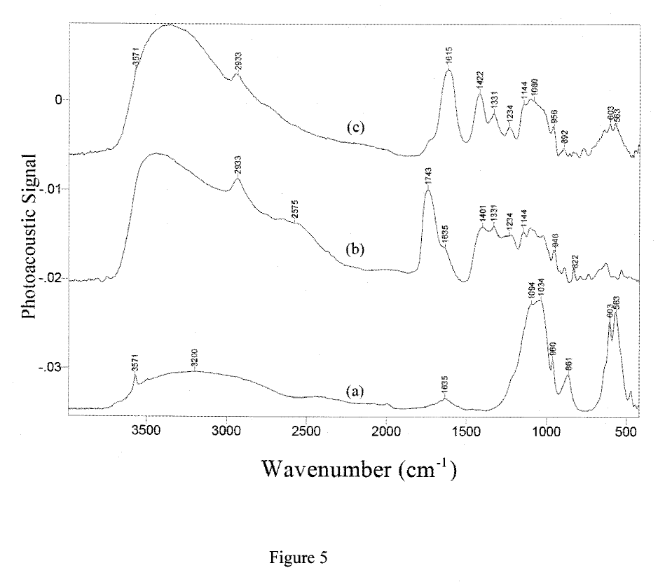

FIG. 5 shows PA-FTIR spectra of HAP, PgA and PgAHAP. The band assignments for chitosan, PgA and hydroxyapatite are described in Table 2 (Synytsya et al., Carbohydrate Polymers 2003; 54:97-106). The band observed at 1743 cm⁻¹ in PgA is assigned to carbonyl stretching of un-dissociated carboxylate groups. The intensity of this band has significantly reduced in PgAHAP. A new band at 1615 cm⁻¹ is observed in PgAHAP. This band originates from asymmetric stretching of dissociated carboxylate groups. While not a limitation, the presence of band due to dissociated carboxylate groups suggest that in PgAHAP, PgA interact to hydroxyapatite through dissociated carboxylate groups. Since carboxylate groups are negatively charged, Ca atoms of HAP are a potential site for attachments. The band at 2933 cm⁻¹ is assigned to C—H stretching vibration and bands at 1331 and 1234 cm⁻¹ are assigned to C—H bending vibrations in the ring. The presence of above bands in PgAHAP indicates that PgA molecular structure is intact in PgAHAP. The band at 2575 cm⁻¹ observed as shoulder to broad OH band at about 3360 cm⁻¹ originate from OH stretching vibration in free carboxyls COOH bonded by hydrogen bonds into dimers. While not a limitation, the absence of this band in PgAHAP composite suggests breaking of PgA dimers. The breaking of dimers is in accordance with dissociation of carboxylate groups.

TABLE 2
Band assignment for polygalacturonic acid

| Band Position cm⁻¹ | Band Assignment |
|---|---|
| 3370 | ν(OH) |
| 2933 | ν(CH) |
| 1743 | ν(C═O)_{COOH} |
| 1635 | δ(H₂O) |
| 1401 | ν, δ(C—OH)_{COOH} |
| 1331 | δ(CH) |
| 1234 | δ(OH)_{COOH} |
| 1144 | ν(COC)_{glycosidic bond, ring} |
| 946 | δ(CCH), δ(COH) |
| 822 | γ(C—OH)_{ring} |

The symbols δ, γ, and ν denote in-plane rocking (r) and scissoring (s), out-of-plane wagging (w) and twisting (t), and stretching modes, respectively.

The band at 1422 cm⁻¹ in PgAHAP composite (FIG. 5) originates from symmetric stretching of carbonyl from dissociated carboxylate groups. The chelation between dissociated groups can be determined from difference in wavenumber between carbonyl asymmetric stretching and symmetric stretching. While not a limitation, the difference of 193 cm⁻¹ suggests unidentate chelation.

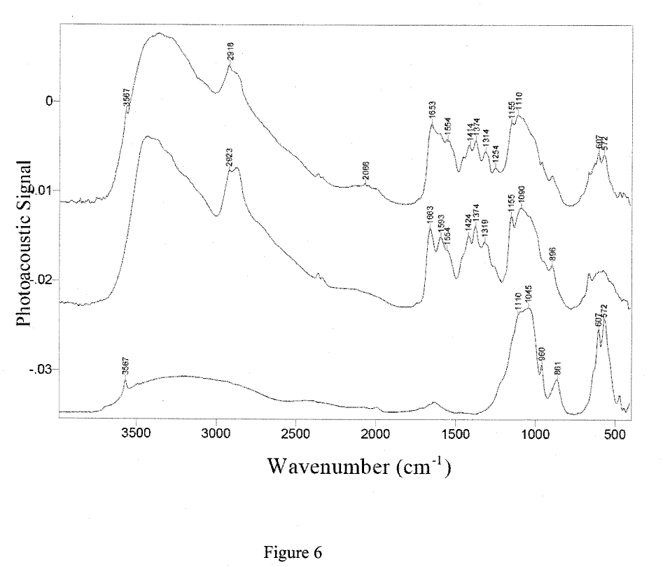

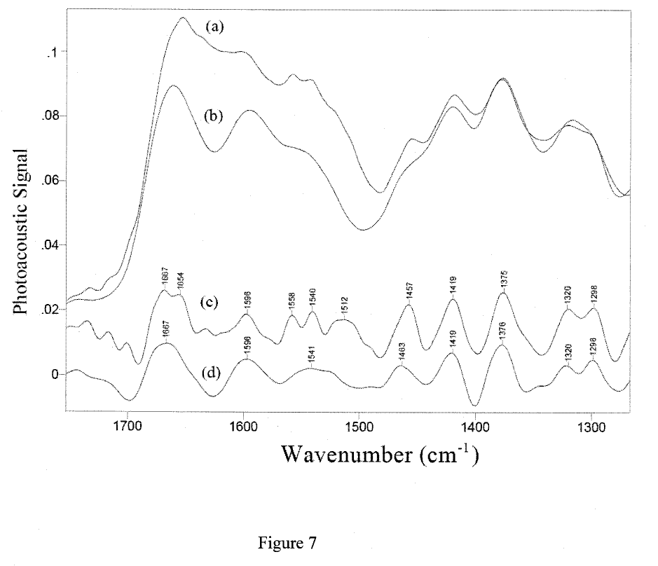

FIG. 6 shows PA-FTIR spectra of HAP, chitosan and ChiHAP. The bands at 1653 and 1319 cm⁻¹ in chitosan are characteristic of N-acetylated chitin and are assigned to amide I and amide II bands. The band assignment for chitosan is given in Table 3 (Pawlak et al., Thermochimica Acta 2003; 396:153-166). The band at 1593 cm⁻¹ in chitosan is assigned to amino characteristic peak. FIG. 7 shows chitosan and ChiHPAP composites spectra in the range of 1800-1200 cm⁻¹. The spectra are normalized with respect to band at 1376 cm⁻¹. This band originates from C—H symmetric deformation. The second derivative plots (FIG. 7) indicates presence of new bands at about 1654 and 1512 cm⁻¹. Presence of these bands in ChiHAP indicates that —NH₂ transform to —NH₃⁺. The bands at 1654 and 1512 cm⁻¹ has been assigned to asymmetric and symmetric deformations of —NH₃⁺ respectively. The presence of NH₃⁺ in ChiHAP is consistent with the presence of a band appearing as shoulder to broad OH band at about 3340 cm⁻¹. This band is assigned to stretching vibration of NH₃⁺. The band at about 2066 cm⁻¹ is also characteristic of NH₃⁺ moieties.

TABLE 3
Band assignments for chitosan

| Band Position cm⁻¹ | Band Assignment |
|---|---|
| 3370 | ν(OH) |
| 2923 | ν(CH) |
| 1663 | Amide I |
| 1593 | Characteristics of amino groups |
| 1374 | δ(CH) |
| 1319 | Amide II |

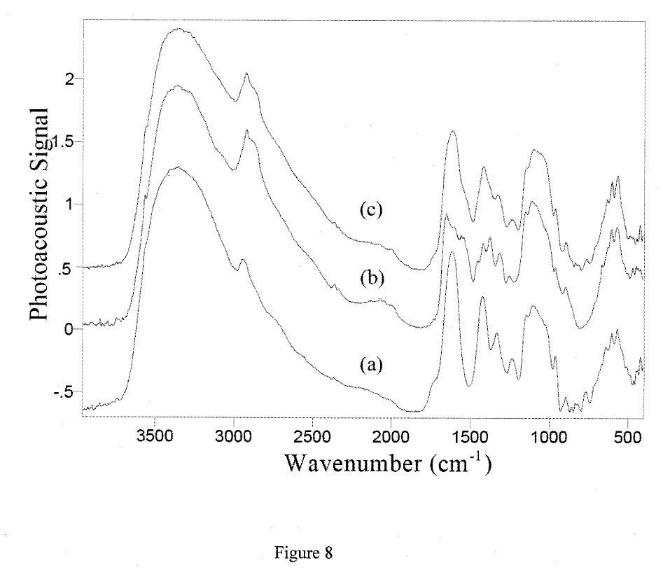

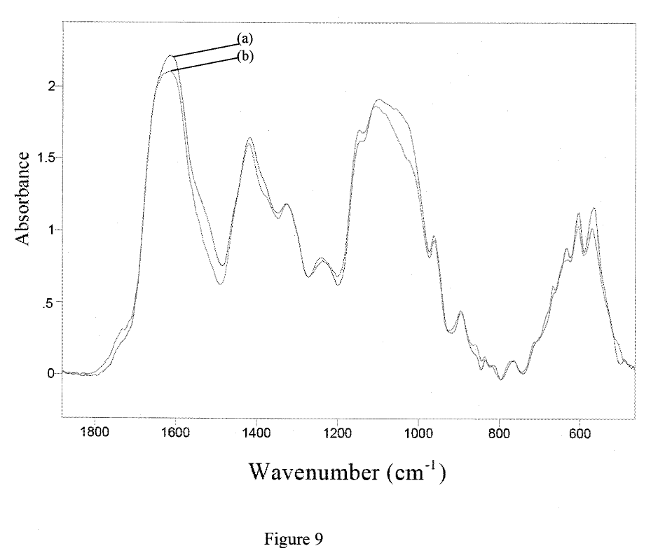

FIG. 8 shows spectra of PgAHAP, ChiHAP and ChiPgAHAP. To have better insight into interfacial interaction in ChiPgAHAP composites, a comparison between the spectrum of ChiPgAHAP and spectrum produced by mathematical addition of ChiHAP and PgAHAP spectra at 50% weightage of each has been done (FIG. 9). While not a limitation, the differences in these spectra suggest that, ChiPgAHAP composite is not simple mixing of ChiHAP and PgAHAP, but there are there are further interfacial interactions happening in the components. It is observed that intensity of band at about 1743 cm⁻¹ is lower in ChiPgAHAP composites and also intensity of band at about 1815 cm⁻¹ and 1422 cm⁻¹ is higher compared to the produced spectrum. Above mentioned observations are clearly indicate that carboxylate groups further dissociate and involve in interfacial interactions between ChiHAP and PgAHAP particles. These dissociated carboxylate groups may interact with hydroxyapatite phase or chitosan functional groups present on surface of ChiHAP particles. From the FIG. 9, it is also observed that, there are changes in phosphate stretching and bending regions. While not a limitation, this suggests that phosphate groups of hydroxyapatite are also involved in interfacial interactions.

### Swelling Behavior Under Simulated Body Fluid (SBF)

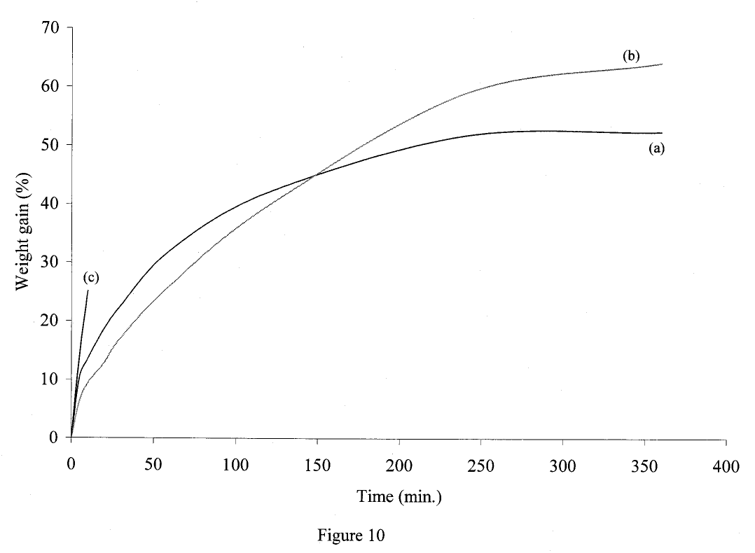

The swelling behavior of the three composites has been studied by soaking samples in SBF. The PgAHAP samples disintegrated in SBF after 10 min. of soaking. The increase in weight of ChiHAP and ChiPgAHAP due to absorption of SBF with time has been plotted in FIG. 10. The SBF absorption has been calculated using following equation:

$$Weight gain ( \% ) = \frac{{W}_{w} - {W}_{d}}{{W}_{d}} \times 100$$

Here, W_{w} is weight of composite after soaking in SBF and W_{d} is dry weight of composites. Both, ChiHAP and ChiPgAHAP maintain their shape even after 24 hours of soaking in SBF. The rate of weight increase of ChiPgAHAP was found to be lower than ChiHAP until 120 min. The maximum weight gain observed in ChiHAP was about 50% and it stabilizes after 4 hrs, on the other hand, ChiPgAHAP continued to gain weight for 6 hours with 62% gain in weight.

The mechanical response and swelling behavior of these composites can be further improved by cross-linking chitosan and PgA together through formation of amide bond between carboxylate and amino groups. As FTIR analysis has shown that at interface chitosan and PgA interact through electrostatic interaction between amino and carboxylate groups. Bernabe et al. have shown that heating chitosan-pectin complex at 120° C. under nitrogen atmosphere, —NH₃⁺⁻OOC bonds can be converted to covalent amide bonds (Bernabe et al., Polymer Bulletin 2005; 55:367-375). The amidation reaction is given by:

—COO⁻+—NH₃⁺→—COHN—

### Materials and Methods

### A. Materials

Na₂HPO₄, ultrapure bioreagent, was obtained from J. T. Baker. CaCl₂, GR grade, was obtained from EM Science. Chitosan and polygalacturonic acid were obtained from Sigma-Aldrich chemicals. All these chemicals and polymers are used as obtained.

### B. In Situ Hydroxyapatite Preparation

Hydroxyapatite (HAP) composites were prepared using a biomimetic process by wet precipitation method (see Verma et al., J Biomed Mater Res. 2006; 77A:59-66; Verma et al., J Biomed Mater Res, 78A, 772-780, 2006; Katti et al., American Journal of Biochemistry and Biotechnology, 2(2), 73-79, 2006). 1 liter of 11.9 mM solution of Na₂HPO₄ using de-ionized water was made. Also prepared was 19.90 mM solution of 1 liter of CaCl₂. Also, 2 grams of PgA was dissolved in 1 liters of Na₂HPO₄ solution. To this solution, 1 liter of CaCl₂ solution was added slowly and subsequently pH of the solution was maintained at 7.4 by adding NaOH. Similarly, hydroxyapatite was also synthesized by dissolving chitosan in 1 liter Na₂HPO₄ solutions prior to addition of CaCl₂ solution. After 12 hours, both solutions were mixed together using magnetic stirrer. Stirring was continued for 12 hours. The schematic for synthesis of chitosan-polygalacturonic acid-hydroxyapatite composite is shown in FIG. 2. After 12 hours, precipitate was separated out by centrifuging. Further, the precipitate was dried in oven at 50° C.

### C. Experimental

X-ray powder diffraction studies of in situ and ex situ HAP was carried out using a Philips diffractometer using CuK_{α} radiation (λ=1.5405 Å). Morphology of porous composites was analyzed using JEOL 6000, JSM 6300V scanning electron microscope. PA-FTIR spectroscopy study was done using Thermo Nicolet, Nexus 870 spectrometer equipped with MTEC Model 300 photoacoustic accessory. Photoacoustic spectra (500 scans) of all samples were collected in the range of 4000-400 cm⁻¹ at a mirror velocity of 0.15 cm/s. AFM-phase imaging was performed using a Multimode AFM having a Nanoscope-IIIa controller equipped with a J-type piezo scanner (Veeco Metrology Group, Santa Barbara, Calif.). Nanoindentation tests were performed using a Triboscope nanomechanical testing instrument (Hysitron Inc., Minneapolis, Minn.) coupled with the Multimode AFM mentioned above.

### EXAMPLE 2

### Composites for Bone Tissue Engineering

Suitability of ChiPgAHAP composites for bone tissue engineering has been evaluated by seeding human osteoblast cells on composite films and scaffolds. This study demonstrates that ChiPgAHAP composites provide a suitable environment for cell adhesion, proliferation and differentiation.

FIG. 11 illustrates that osteoblast cells form nodular structure, which is a sign of cell differentiation. The mineralization or calcium deposition in these nodules has been verified by staining these nodules with alizarin red S.

FIG. 12 illustrates osteoblast cells seeded in scaffold and stained with Live/Dead cell assay. Positive green stain shows that cells are alive after 21 days from seeding.

FIG. 13 illustrates SEM images of fibrous extracellular matrix synthesized by osteoblast on ChiPgAHAP films.

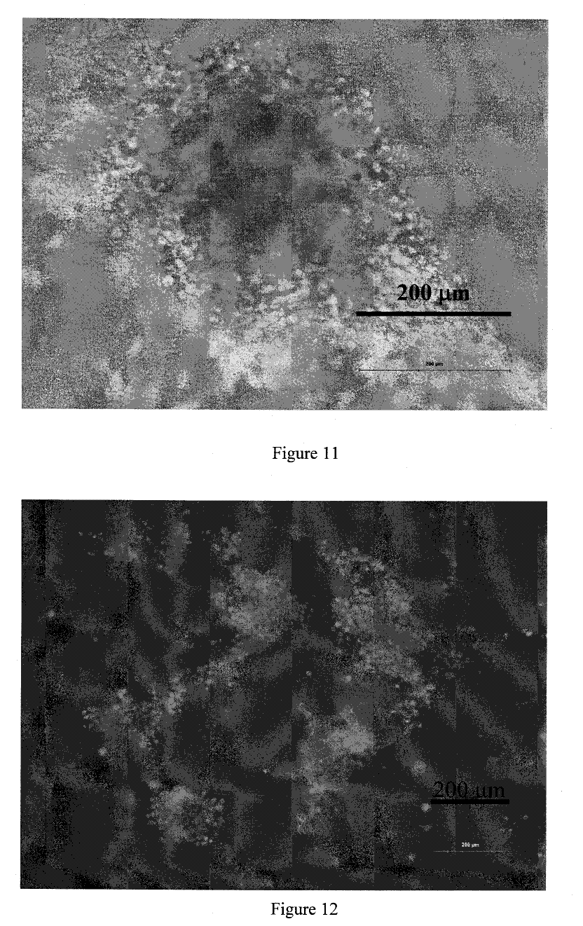

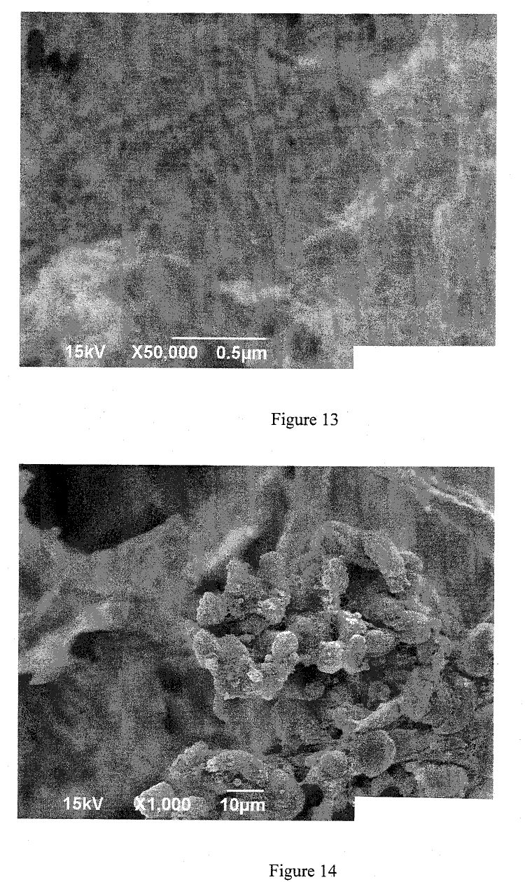

FIG. 14 illustrates proliferation of osteoblast cells and matrix formation.

### EXAMPLE 3

### Effect of Biopolymers on Structure of Hydroxyapatite and Interfacial Interactions in Biomimetically Synthesized Hydroxyapatite/Biopolymer Nanocomposites

The interfacial interaction and effect of biopolymer on crystal structure of hydroxyapatite in biomimetically synthesized nanocomposites, chitosan/hydroxyapatite (ChiHAP), polygalacturonic acid/hydroxyapatite (PgAHAP) and chitosan/polygalacturonic acid/hydroxyapatite (ChiPgAHAP) have been investigated using atomic force microscopy (AFM), Fourier transform infrared (FTIR) spectroscopy and Rietveld analysis. AFM phase images show nano sized hydroxyapatite particles uniformly distributed in biopolymer. FTIR spectra indicate that chitosan interacts with hydroxyapatite through NH₃⁺ groups, whereas in polygalacturonic acid/hydroxyapatite, dissociated carboxylate groups (COO⁻) form unidentate chelate with calcium atoms. A change in lattice parameters of hydroxyapatite in all nanocomposites is observed using Rietveld analysis. The increase in lattice parameters was most prominent along c-axis in ChiHAP and ChiPgAHAP nanocomposites, which was 0.388% and 0.319% respectively. Comparison between particle sizes of hydroxyapatite, determined from AFM and Rietveld analysis, indicates presence amorphous phase in hydroxyapatite particles, which is believed to be present at the interface of hydroxyapatite and biopolymer.

Organisms produce complex structures containing minerals with controlled size, shape, crystal orientation, polymorphic structure, defect texture, and particle assembly. This process of mineralization in organisms is called as biomineralization. Biomineralization is a complex process, which is controlled by organisms by secretion of various organics, mainly proteins.

As described herein, composites of chitosan, polygalacturonic acid and hydroxyapatite were synthesized. The composites were synthesized by allowing precipitation of hydroxyapatite in presence of biopolymers. These nanocomposites were developed for their possible application as bone biomaterials. Three kinds of hydroxyapatite/biopolymer composites were made: chitosan/hydroxyapatite (ChiHAP), polygalacturonic acid/hydroxyapatite (PgAHAP) and chitosan/polygalacturonic acid/hydroxyapatite (ChiPgAHAP). Chitosan and polygalacturonic acid are electrostatically complementary to each other. The strong electrostatic interaction between these two biopolymers is believed to have led to enhancement of the mechanical response in chitosan/polygalacturonic acid/hydroxyapatite composites.

The particle size, shape and interfacial interactions in a biomaterial have significant impact on its mechanical response, biocompatibility and biodegradability. As described, the effect of biopolymers on crystallite structure, crystal size and interfacial interactions were investigated using atomic force microscopy (AFM), X-ray diffraction (XRD) and Fourier transform infrared (FTIR) spectroscopy.

### Materials and Methods

### A. Materials

Na₂HPO₄, ultrapure bioreagent, was obtained from J. T. Baker. CaCl₂, GR grade, was obtained from EM Science. Chitosan and polygalacturonic acid were obtained from Sigma-Aldrich chemicals. All these chemicals and polymers are used as obtained

### B. Hydroxyapatite and Hydroxyapatite/Biopolymer Composites Preparation

The nanocomposites were synthesized using a biomimetic method. The details of synthesis method have been given elsewhere. Briefly, ChiHAP and PgAHAP were synthesized by dissolving chitosan and PgA in Na₂HPO₄ solution separately. Later, CaCl₂ solution was added and pH was maintained at 7.4 by adding NaOH solution. The precipitates were allowed to settle for 24 hours. Further, water was removed by centrifuging followed by drying at 50° C. ChiPgAHAP was synthesized by mixing together Na₂HPO₄ solutions containing chitosan and PgA in 1:1 ratio. Further, precipitates were removed by centrifuging and drying at 50° C.

### C. Experimental

X-ray powder diffraction studies of hydroxyapatite and hydroxyapatite/biopolymer composites were carried out using a Philips diffractometer using CuK_{α} radiation (λ=1.5405 Å). PA-FTIR spectroscopy study was done using Thermo Nicolet, Nexus 870 spectrometer equipped with MTEC Model 300 photoacoustic accessory. Linear photoacoustic spectra (500 scans) of all samples were collected in the range of 4000-400 cm⁻¹ at a mirror velocity of 0.15 cm/s. AFM-phase imaging was performed using a Multimode™ AFM having a Nanoscope-IIIa™ controller equipped with a J-type piezo scanner (Veeco Metrology Group, Santa Barbara, Calif.).

### D. Structure Refinement

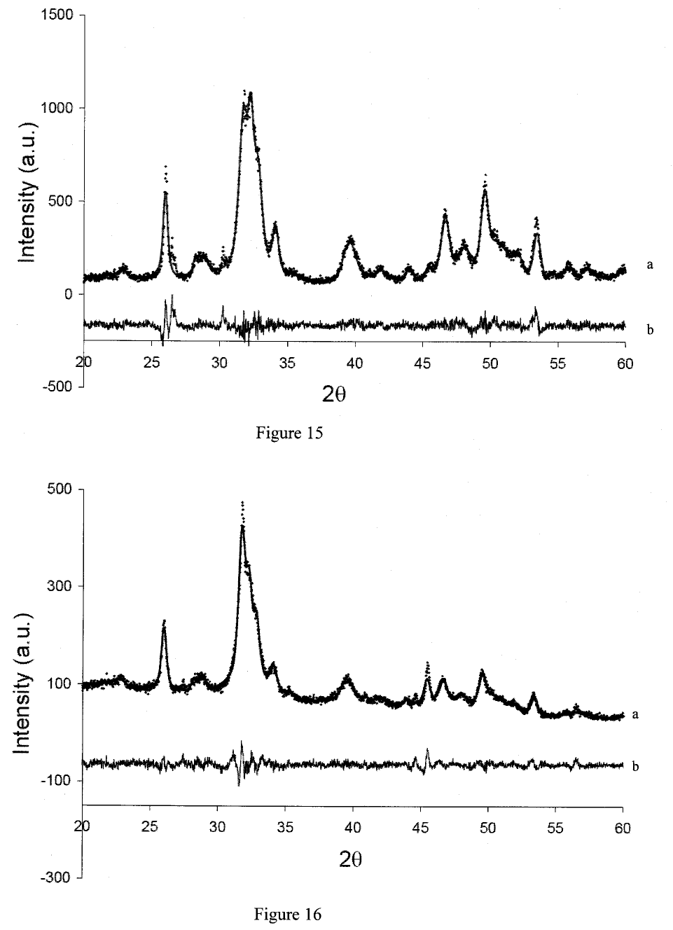

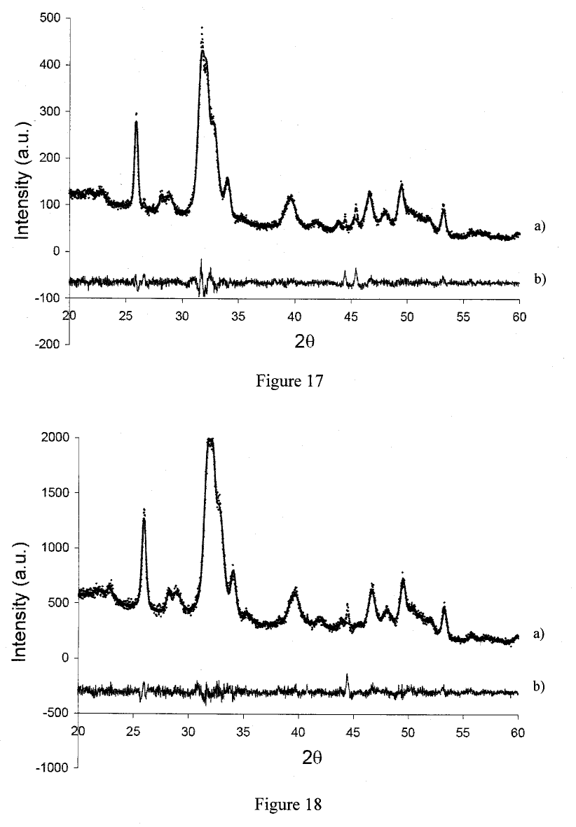

Rietveld analysis of pure hydroxyapatite and its composites with chitosan, polygalacturonic acid and both chitosan and polygalacturonic acid were performed using Reflex™ module of Materials Studio™ (Accelrys Software Inc.) Software. FIGS. 15-18 show x-ray diffraction plots of hydroxyapatite, ChiPgAHAP, ChiHAP and PgAHAP composites. The refinement was done in the range of 20-60° 2θ. The lattice parameters of starting model were a=b=9.424 Å and c=6.879 Å with P6₃/M space group. The occupancy factor of all atoms was fixed to 1 except for OH. The occupancy factor for OH was fixed at 0.5. The profile function used was Pseudo-Voigt. The temperature factors were refined using atomic anisotropic parameters. The sample parameters: crystallite size, lattice strain and preferred orientation were also refined. Preferred orientation was refined using March-Dollase function.

### Result and Discussion

### Nanostructure Analysis Using Atomic Force Microscopy (AFM)

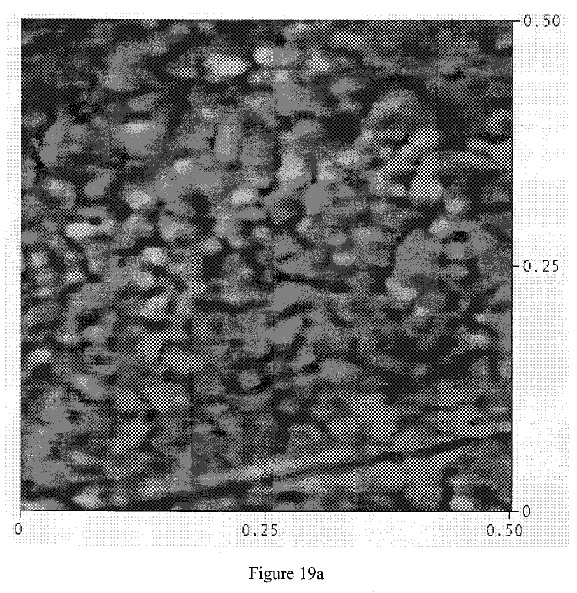

FIG. 19 (19a, 19b, 19c) shows AFM-phase images of PgAHAP, ChiHAP, and ChiPgAHAP composites. The HAP and biopolymer phases are clearly distinguishable from AFM-phase images. In PgAHAP, ChiHAP and ChiPgAHAP, the average HAP particle size are 25 nm, 43 nm and 34 nm respectively. The smaller particle size of hydroxyapatite in PgAHAP may be attributed to inhibitory effect caused by carboxylate groups of polygalacturonic acid.

### Interfacial Interactions

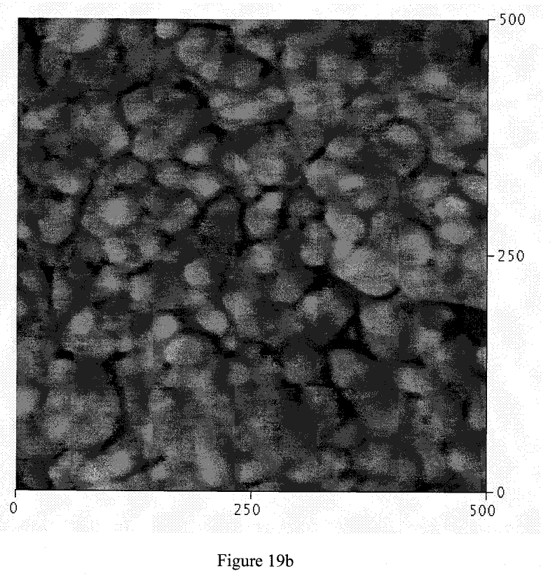

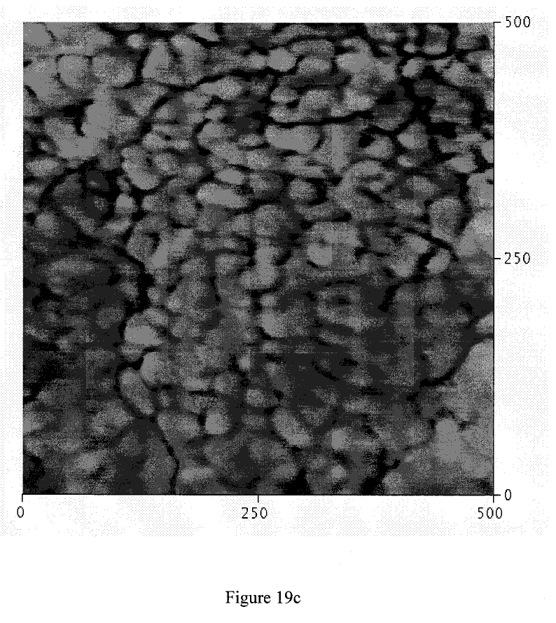

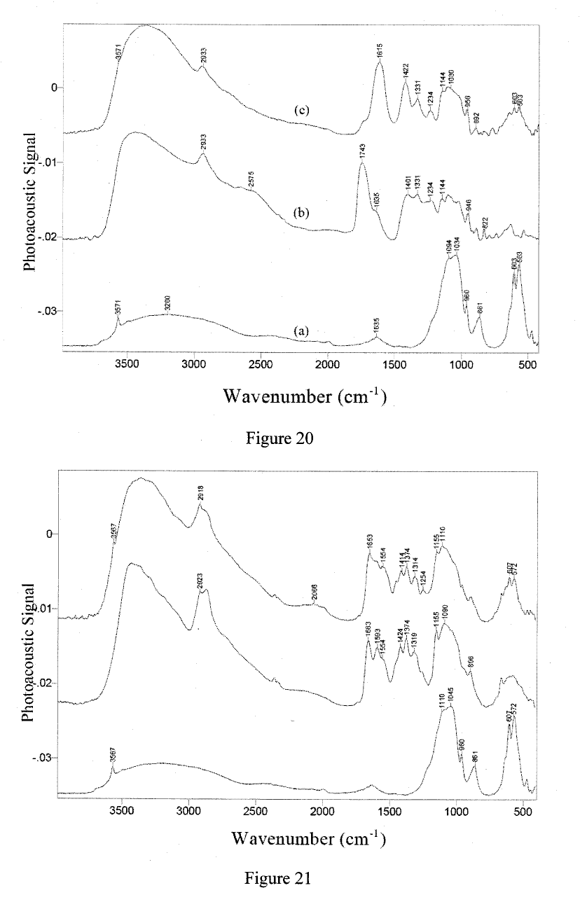

FIG. 20 shows PA-FTIR spectra of HAP, PgA and PgAHAP. The band observed at 1743 cm⁻¹ in PgA is assigned to carbonyl stretching of un-dissociated carboxylate groups. The intensity of this band is significantly reduced in PgAHAP as compared to PgA. A new band at 1615 cm⁻¹ is observed in PgAHAP. This band is attributed to asymmetric stretching of dissociated carboxylate groups. The presence of band due to dissociated carboxylate groups suggest that in PgAHAP, PgA interacts with hydroxyapatite through dissociated carboxylate groups. Since carboxylate groups are negatively charged, Ca atoms of HAP may act as potential sites for attachments.

The band at 2933 cm⁻¹ is assigned to C—H stretching vibration and bands at 1331 and 1234 cm⁻¹ are assigned to C—H bending vibrations of the ring. The band at 2575 cm⁻¹, observed as shoulder to the broad OH band at around 3360 cm⁻¹ originates from OH stretching vibration in free carboxyls bonded by hydrogen bonds into dimers. The absence of this band in PgAHAP composite suggests breaking of PgA dimers. The breaking of dimers is consistent with the observation of dissociation of carboxylate groups. The band at 1422 cm⁻¹ in PgAHAP composite (FIG. 6) originates from symmetric stretching of carbonyl from dissociated carboxylate groups. The chelation between dissociated groups can be determined from difference in wavenumber between carbonyl asymmetric stretching and symmetric stretching. Here, a difference of 193 cm⁻¹ suggests unidentate chelation.

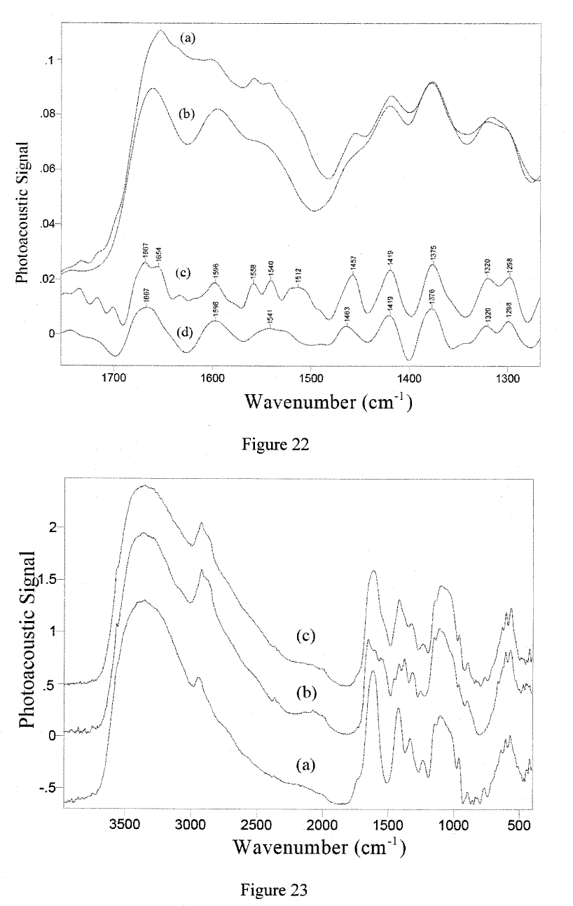

FIG. 21 shows PA-FTIR spectra of HAP, chitosan and ChiHAP. The bands at 1653 cm⁻¹ and 1319 cm⁻¹ in chitosan are characteristic of N-acetylated chitin and are assigned to amide I and amide II bands. The band at 1593 cm⁻¹ in chitosan is assigned to amino characteristic peak. FIG. 21 shows chitosan and ChiHPAP composites spectra in the range of 1800-1200 cm⁻¹. The spectra are normalized with respect to band at 1376 cm⁻¹. This band originates from C—H symmetric deformation. The second derivative plots (FIG. 22) suggest presence of new bands at around 1654, 1558 and 1512 cm⁻¹. Presence of these bands in ChiHAP indicates that —NH₂ transforms to —NH₃⁺. The bands at 1654 cm⁻¹ and 1580 cm⁻¹ have been assigned to asymmetric deformation and band at 1512 cm⁻¹ is assigned to symmetric deformations of —NH₃⁺. The presence of NH₃⁺ in ChiHAP is consistent with the presence of a band appearing as shoulder to the broad OH band at around 3340 cm⁻¹. This band is assigned to stretching vibration of NH₃⁺. The band at around 2066 cm⁻¹ is also characteristic of NH₃⁺ moieties. The NH₃⁺ functional groups being positively charged, forms complex with phosphate ions. This complex further facilitates nucleation and growth of hydroxyapatite.

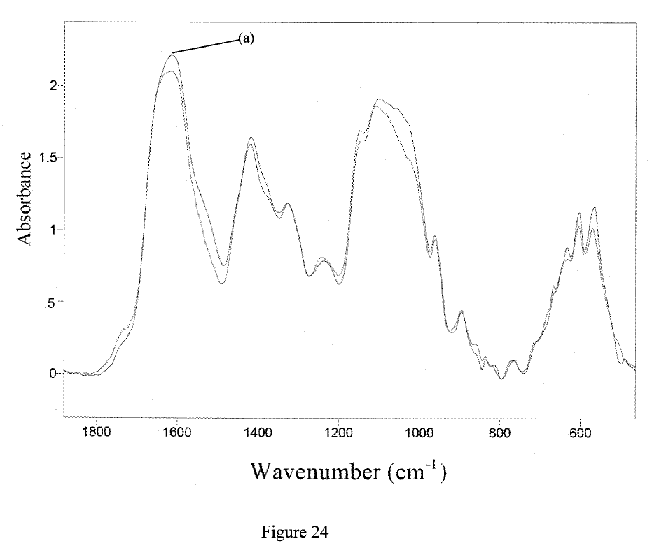

FIG. 23 shows spectra of PgAHAP, ChiHAP and ChiPgAHAP. To have a better insight into interfacial interaction in ChiPgAHAP composites, a comparison between the photoacoustic FTIR spectrum of ChiPgAHAP and the spectrum obtained by addition of ChiHAP and PgAHAP spectra with 50% weightage of each has been done (FIG. 24). As seen, the comparison between these spectra suggests that, ChiPgAHAP composite does not result from simple mixing of ChiHAP and PgAHAP, but there are there are further interfacial interactions occurring in the composite. The intensity of band at around 1743 cm⁻¹ is lower in ChiPgAHAP composites and also intensity of band at around 1815 cm⁻¹ and 1422 cm⁻¹ is higher compared to mathematically added spectrum. The above mentioned observations clearly indicate that carboxylate groups further dissociate and are involved in interfacial interactions between ChiHAP and PgAHAP particles. These dissociated carboxylate groups may interact with hydroxyapatite phase or chitosan functional groups present on surface of ChiHAP particles. It is also observed that there are changes in phosphate stretching and bending regions. This suggests that phosphate groups of hydroxyapatite are also involved in interfacial interactions.

### Effect of Biopolymers on Structure of Hydroxyapatite

Hydroxyapatite crystallizes in a hexagonal crystal lattice with P6₃/m space group (Haverty et al., Physical Review B—Condensed Matter and Materials Physics. 71:1-9, 2005). The lattice parameters calculated from Rietveld refinement are given in Table 3. The Rietveld analysis indicates that there is a change in lattice parameters of hydroxyapatite present in biopolymer/hydroxyapatite nanocomposites as compared to pure hydroxyapatite. The biomimetic hydroxyapatite showed similar trend in all nanocomposites i.e., positive shift (elongation) was observed along c-axis whereas negative shift (contraction) was observed along a-axis (a=b). The elongation along c-axis was significantly higher in ChiHAP and ChiPgAHAP than PgAHAP (Table 4). This large change is rather significant in a ceramic material such as HAP. The composites consist of nano-sized HAP particles and hence a very large surface interacts with biopolymers. This large interfacial interaction may lead to large lattice distortions of hydroxyapatite. The shift in ChiHAP and ChiPgAHAP was significantly higher than PgAHAP composites, which suggest that the shift in lattice parameters also depend on the type of biopolymers.

TABLE 3
Lattice parameters determined from Rietveld analysis XRD data.
The (↓) symbol depicts decrease in lattice parameter of biomimetic
hydroxyapatite with respect to hydroxyapatite and (↑) symbol
depicts increase in lattice parameters of biomimetic hydroxyapatite
with respect to pure hydroxyapatite.

| | HAP | ChiHAP | PgAHAP | ChiPgAHAP |
|---|---|---|---|---|
| | (Å) | (Å) | (Å) | (Å) |
| a, b | 9.464 | 9.460 ↓ | 9.453 ↓ | 9.455 ↓ |
| c | 6.856 | 6.882 ↑ | 6.858 ↑ | 6.878 ↑ |

TABLE 4
The percentage change in lattice parameters of biomimetic
hydroxyapatite with respect to pure hydroxyapatite.

| | ChiHAP | PgAHAP | ChiPgAHAP |
|---|---|---|---|
| a, b | −0.019% | −0.085% | −0.068% |
| c | +0.388% | +0.039% | +0.319% |

These results indicate that crystal distortions by organics can be achieved by following a biomimetic synthesis method. The spatial mismatch between functional groups of biopolymer and hydroxyapatite lattice sites can cause residual stresses at the biopolymer/mineral interface. This residual stress can lead to distortion in the crystal structure of hydroxyapatite. The second phenomena, which could cause stress on crystal lattice, is the conformational change in biopolymers, while compaction of composites during drying.

Also, it appears that the biopolymers are not only affecting crystal structure of hydroxyapatite but they are also affecting their crystallinity. The crystallite sizes calculated from Rietveld analysis are found to be smaller compared to particle size determined from AFM-phase images (Table 5). This suggests that either HAP particles observed from AFM-phase images have multi-granular structure or they contain significant amount of amorphous phase. The difference between particle size and their respective crystallite size is more pronounced in ChiHAP and ChiPgAHAP. The growth of hydroxyapatite particles in presence of chitosan or PgA resemble the biomineralization process, where proteins and other biopolymers control the growth of the mineral.

TABLE 5
crystallite sizes of biomimetic hydroxyapatite from Rietveld analysis
using XRD data.

| | HAP | ChiHAP | PgAHAP | ChiPgAHAP |
|---|---|---|---|---|
| | (nm) | (nm) | (nm) | (nm) |
| a, b | 11.1 | 17.2 | 23.9 | 10.0 |
| c | 17.1 | 28.5 | 16.4 | 16.5 |

The functional sites of biopolymers act as a nucleating sites for crystallization of mineral. The negatively charged carboxylate groups of polygalacturonic acid are known for their calcium binding ability. The dissociated carboxylate groups and calcium ions form a complex, which initiate further growth of hydroxyapatite (Bhowmik et al., Mat. Sci. Eng C. 27:352-371, 2007; and Verma et al., Journal of Biomedical Materials Research Part A, 77A:59-66, 2006). On the other hand, in case of chitosan, amine being negatively charged, form complex with phosphate groups. Because these nucleating sites in chitosan or polygalacturonic acid are not spatially arranged and oriented to have perfect match with lattice sites of phosphate or calcium ions, it is most likely that hydroxyapatite precipitate as amorphous phase near polymer surface (FIG. 25).

### CONCLUSIONS

The interfacial molecular interactions between biopolymers and hydroxyapatite have been investigated. Also analyzed was the effect of biopolymer on structure of hydroxyapatite in biomimetic composites using Rietveld analysis. The intermolecular interactions between different components have been studied using Fourier transform infrared (FTIR) spectroscopy. The FTIR spectra indicate that in PgAHAP, polygalacturonic acid attaches to hydroxyapatite surface through dissociated carboxylate groups, whereas in ChiHAP, chitosan interacts with hydroxyapatite through NH groups. The FTIR results also indicate that in ChiPgAHAP composites, chitosan, PgA and hydroxyapatite, all participate in interfacial interactions. The AFM phase images indicate a uniform dispersion of hydroxyapatite nanoparticles in biopolymer matrix. A comparison between hydroxyapatite particle size determined from AFM imaging and Rietveld analysis, suggest either hydroxyapatite particles have multi-granular structure or contain significant amount of amorphous phase. This amorphous phase is believed to be present at the interface of hydroxyapatite and biopolymer. Rietveld analysis has also shown a change in lattice parameters of biomimetic hydroxyapatite. The shift in lattice parameters was found to be highest along c-axis in chitosan containing (ChiHAP and ChiPgAHAP) nanocomposites. A change of 0.4% in the structure of hydroxyapatite is observed. This is a rather significant change resulting from nanoscale interaction of biopolymer and mineral. It is expected that such weak interactions also occur in many biological and bio-replacement materials which result in significant changes to lattice structure of mineral.

### EXAMPLE 4

### A Synthetic Procedure for Biopolymer-HAP Fiber Formation

In this example, the chitosan solution was prepared by dissolving 0.1 g chitosan in 100 ml of deionized water, and the PgA solution was prepared by dissolving 0.1 g PgA in 100 ml of deionized water. These two solutions were mixed together by adding, drop-wise, the chitosan solution to the PgA solution. The mixed solution was sonicated and freeze dried. The freezing of the solution was by immersing the beaker containing the solution into a liquid nitrogen containing bath.

The ChiPgAHAP composite fibrous scaffold was made by adding a HAP solution to the ChiPgA solution prior to freezing. After addition of HAP, the resulting solution was further sonicated for proper mixing.

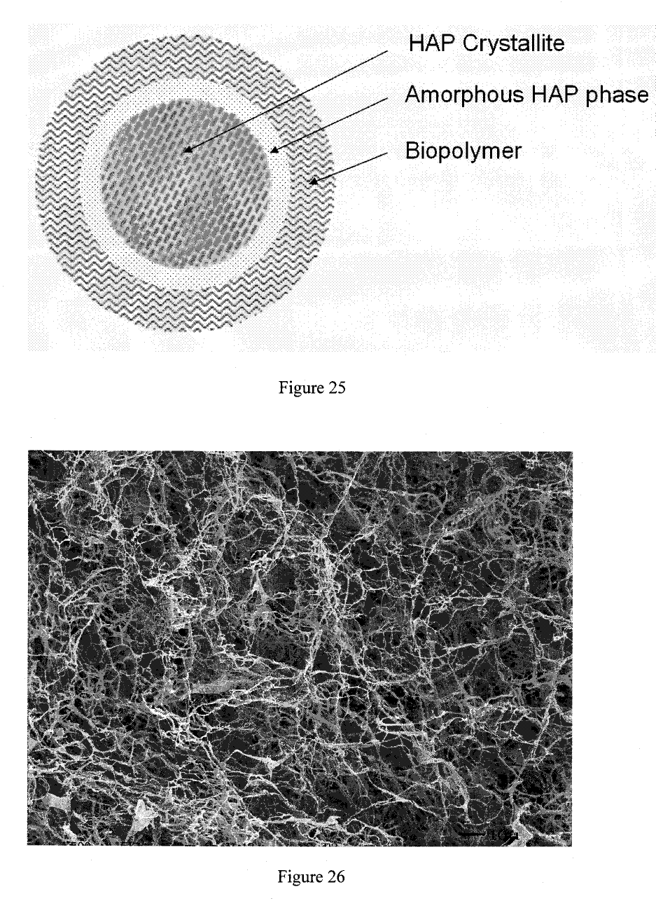

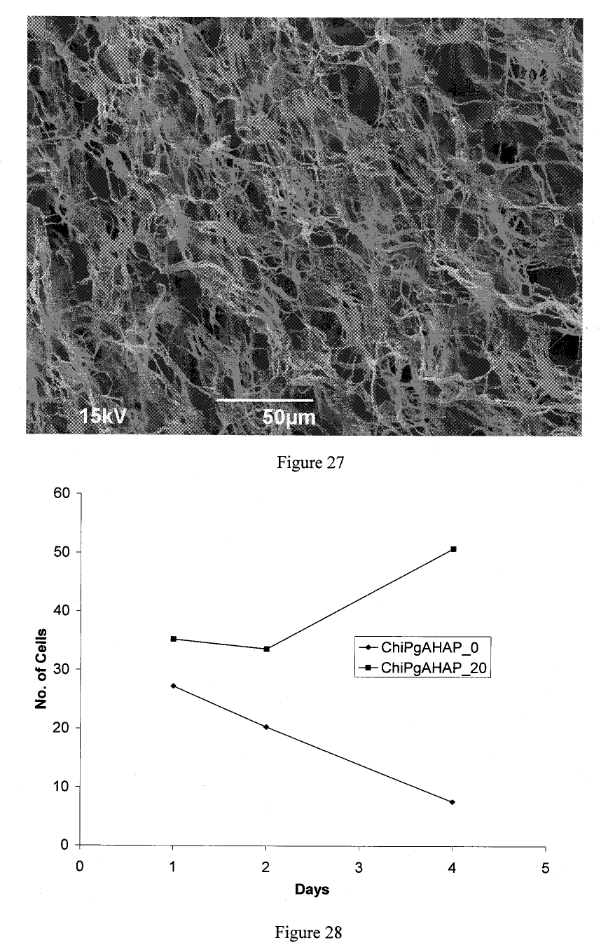

SEM Images of the ChiPgA and ChiPgAHAP scaffolds are shown in FIGS. 26 and 27. Both types of fibers are 1-2 μm in diameter. Biocompatibility studies are presented in FIG. 28 that indicate a higher biocompatibility results from addition of hydroxyapatite mineral in fibers. Mechanical tests are underway.

All publications, patents and patent applications cited herein are incorporated herein by reference.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composite comprising chitosan, polygalacturonic acid, and hydroxyapatite.

2. The composite of claim 1, wherein the composite is in the form of a fibrous scaffold.

3. The composite of claim 2, wherein the fibers of the fibrous scaffold are about 1-2 μm in diameter.

4. The composite of claim 1, wherein the composite is in the form of a film.

5. The composite of claim 1, wherein the composite further comprises osteoblast cells.

6. The composite of claim 1, wherein the composite further comprises calcium mineralization.

7. A method for preparing a composite that comprises chitosan, polygalacturonic acid, and hydroxyapatite, comprising:
preparing a first mixture that comprises chitosan and hydroxyapatite so as to form a composite that comprises chitosan and hydroxyapatite;
preparing a second mixture that comprises polygalacturonic acid and hydroxyapatite so as to form a composite that comprises polygalacturonic acid and hydroxyapatite; and
combining the first and second mixtures to form a third mixture so as to form a composite that comprises chitosan, polygalacturonic acid, and hydroxyapatite.

8. The method of claim 7, wherein the hydroxyapatite in the first mixture is prepared by combining Na₂HPO₄ and CaCl₂ so as to form the hydroxyapatite.

9. The method of claim 7, wherein the hydroxyapatite in the second mixture is prepared by combining Na₂HPO₄ and CaCl₂ so as to form the hydroxyapatite.

10. The method of claim 7, further comprising cross-linking the chitosan to the polygalacturonic acid.

11. The method of claim 7, further comprising separating the composite that comprises chitosan, polygalacturonic acid, and hydroxyapatite from the third mixture.

12. The method of claim 7, further comprising drying the composite that comprises chitosan, polygalacturonic acid, and hydroxyapatite.

13. A method for preparing a composite that comprises chitosan, polygalacturonic acid, and hydroxyapatite, comprising:
preparing a first mixture that comprises chitosan in deionized water;
preparing a second mixture that comprises polygalacturonic acid in deionized water;
combining the first and second mixtures to form a third mixture; and
combining a fourth mixture that comprises hydroxyapatite with the third mixture to form a fifth mixture so as to form a composite that comprises chitosan, polygalacturonic acid, and hydroxyapatite.

14. The method of claim 13, further comprising sonicating the third mixture.

15. The method of claim 13, further comprising freezing the fourth mixture.

16. The method of claim 13, further comprising sonicating the fifth mixture.

17. A composite that comprises chitosan, polygalacturonic acid, and hydroxyapatite prepared according to the method of claim 7.

18. The composite of claim 17, wherein the composite is in the form of a fibrous scaffold.

19. The composite of claim 18, wherein the fibers of the fibrous scaffold are about 1-2 μm in diameter.

20. The composite of claim 17, wherein the composite is in the form of a film.

21. The composite of claim 17, wherein the composite further comprises osteoblast cells.

22. The composite of claim 17, wherein the composite further comprises calcium mineralization.

23. A method for preparing a composite, comprising preparing a mixture that comprises chitosan, Na₂HPO₄ and CaCl₂ so as to form a composite that comprises chitosan and hydroxyapatite.

24. A method for preparing a composite, comprising preparing a mixture that comprises polygalacturonic acid, Na₂HPO₄ and CaCl₂ so as to form a composite that comprises polygalacturonic acid and hydroxyapatite.

25. A composite prepared according to the method of claim 23.

26. A method for treating a patient having a damaged bone, comprising inserting into the patient the composite of claim 1 so as to treat the damaged bone.

27. The method of claim 26, wherein the bone was damaged by an injury.

28. The method of claim 26, wherein the bone was damaged by a disease.

29. The method of claim 26, wherein the damaged bone is a portion of a joint.

30. A composition comprising a composite as described in claim 1 and an acceptable carrier.

31. The composition of claim 30, wherein the carrier is a pharmaceutically acceptable carrier.

32-37. (canceled)

38. A composite that comprises chitosan, polygalacturonic acid, and hydroxyapatite prepared according to the method of claim 13.

39. A composite prepared according to the method of claim 24.
